(19) United States
(12) Patent Application Publication
(10) Pub. No.: US 2007/0203040 A1
(43) Pub. Date: Aug. 30, 2007

(54) Bar soap

(76) Inventors: Harry Reicherz, Rutherford, NJ (US); Stephen Heydt, Wayne, NJ (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/708,952

(22) Filed: Feb. 21, 2007

Related U.S. Application Data
(60) Provisional application No. 60/776,761, filed on Feb. 24, 2006.

(51) Int. Cl. A61K 8/02

(52) U.S. Cl. 510/141

(57) ABSTRACT

The present disclosure provides a bar soap for treating uroshiol induced dermatitis including an alkylphenol ethoxylate, a sulfur-containing surfactant, a soap and water.

### CROSS-REFERENCE TO RELATED APPLICATION

The present application claims the benefit of and priority to U.S. Provisional Application Ser. No. 60/776,761, filed on Feb. 24, 2006, the entire disclosure of which is incorporated herein by reference.

### BACKGROUND

1. Technical Field

The present disclosure relates generally to bar soap. More particularly, this disclosure relates to bar soap for treating uroshiol-induced dermatitis.

2. Background of Related Art

Urushiol is the toxin responsible for the allergic dermatitis caused by contact with the sap of commonly encountered noxious plants such as poison ivy, poison oak, and poison sumac, and related plants found throughout the world. Urushiol or related chemicals are also found in the Anacardiaceae group, which includes, among others, the lacquer tree of Asia, mango tree, cashew shell oil, and in certain nut shells, such as the walnut.

Chemically, urushiols are mixtures of catechols with long, hydrophobic, carbon(alkyl) side chains at the three position of the catechol ring. For example, poison ivy contains predominantly 3-n-pentadececylcatechols (C-15) and poison oak contains predominantly 3-n-heptaecylcatechols (C-17). When located inside an unruptured plant leaf, Urushiol is a light, colorless oil. When exposed to oxygen, urushiol easily oxidizes and, after polymerizing, turns a blackish color.

The allergic reaction is the result of exposure to the oleoresin containing the urushiol. The reaction is an allergic eczematous contact dermatitis characterized by redness, swelling, papules, vesicles, bullac, and streaking.

Treatment has historically consisted of attempting to remove the oil as quickly after exposure as possible: applying rubbing alcohol, washing effected areas with water, and showering with soap and water. Unfortunately, if the above procedure is not commence within minutes of exposure the regimen will not remove the toxin but may limit its spread.

Attempts have been made to find both prophylactic treatments as well as post-exposure treatments. To date, no vaccine has been developed and the prior art treatments are not without shortcomings. One treatment example is seen in U.S. Pat. No. 5,686,074 to Stewart which teaches and claims a treatment for poison ivy which includes a composition including linseed oil, an astringent, a starch, an essential oil and a citrus oil. One shortcoming of this patent is that linseed oil can cause irritation itself. A second shortcoming of this patent is that it requires that the composition be applied to the effected areas up to twice a day until the rash is gone. The composition provides what appears to be only very temporary palliative relief of poison ivy symptoms and does not appear to alter the course of the malady.

Yet other prior art attempts have focused on prophylactics for preventing the dermatitis. One example is seen is U.S. Pat. No. 4,663,151 to Waali which discloses and claims a prophylactic treatment based upon Aluminum Chlorhydrate. Of course, the most significant shortcoming associated with prophylactic treatments is that they are only effective if applied before exposure to the urushiol; an occurrence that rarely takes place.

There is a need, therefore, for a safe, effective treatment for dermatitis caused by exposure to the toxin urushiol. Despite the prior art, the known solutions are still not entirely satisfactory. The treatment should provide complete relief from the signs and symptoms associated with the dermatitis in limited treatments and be effective at any point during the dermatitis cycle.

### SUMMARY

The present disclosure relates to a bar soap used for treating uroshiol-induced dermatitis, which includes an alkyl phenol ethoxylate, a sulfur-containing surfactant, a soap and water. Additional ingredients may be added to the bar soap to improve the soaps consistency, lather, scent, and rinsability, as well as maintain or improve the ability of the soap bar to treat uroshiol-induced dermatitis.

Also, in accordance with the present disclosure, a method of treating uroshiol-induced dermatitis is described including preparing a bar soap which includes an alkylphenol ethoxylate, a sulfur-containing surfactant, a soap and water, washing the affected area with the bar soap and rinsing of the affected area to remove the uroshiol.

### DETAILED DESCRIPTION

The present disclosure relates to bar soaps useful for treating uroshiol-induced dermatitis that contain an alkylphenol ethoxylate, a sulfur-containing surfactant, a soap and water.

Alkylphenols are formed during the alkylation process of phenols, particularly in the synthesis of polyethoxylate detergents. In alkylphenols, a hydrocarbon chain of 1-20 carbon atoms is attached to the phenol ring in either the ortho (2), meta (3), or para (4) position, with the most common ring isomers being ortho or para. Moreover, the alkyl chains can exist as either linear n-alkyl chains, or complex branched chains. Some commonly known alkyl phenols include, but are not limited to, ethylphenol, butylphenol, octylphenol, and nonylphenol.

Ethoxylated alkylphenols, alkylphenol ethoxylates (APE), are formed through the process of ethoxylation. Ethoxylation is a chemical process in which ethylene oxide (IUPAC name: 1,2-epoxyethane) is added to fatty acids, alcohols, amines or phenols, i.e., specifically the alkylphenols as described herein, in order to make them more soluble in water.

It is envisioned that any alkylphenol ethoxylate suitable for forming a bar soap can be used. In particularly useful embodiments, the alkylphenol ethoxylate is nonoxynol.

It is envisioned that the ethoxylated alkylphenol may represent from about 1.0 to about 50% by total weight of the bar soap. In embodiments, the ethoxylated alkylphenol may represent from about 5% to about 45% by total weight of the bar soap.

The second component of the bar soap described herein includes at least one sulfur-containing surfactant. Suitable examples of useful sulfur-containing surfactants include, but are not limited to, alkyl sulfates, sodium dodecyl sulfate, sodium lauryl sulfate, sulfoacetates, alkyl ether sulfates, oxyalkyl ether sulfates, linear alkylbenzene sulfonates, secondary alkane sulfonates, sulfosuccinates, sodium sulfosuccinate, disodium lauryl sulfosuccinate, disodium laureth sulfosuccinate, sodium coco sulfate, α-olefine sulfonates, sulfonated fatty-acids and combinations thereof. The most widely used surfactants include the sodium salt, but raw materials with various other cations like, e.g., ammonium, magnesium may be used such as ammonium lauryl sulfate or magnesium lauryl sulfate.

It is envisioned that the sulfur-containing surfactant may represent from about 0.1 to about 40% by total weight of the bar soap. In embodiments, the sulfur-containing surfactant may represent from about 1.0 to about 35% by total weight of the bar soap.

The third component used in forming the bar soaps described herein includes at least one soap. In some embodiments, soaps corresponding to the following formula:
R¹CO—ONa
in which R¹CO is an aliphatic acyl radical containing 4 to 22 carbon atoms are suitable for use in accordance with the invention. Typical examples are the sodium salts of caproic acid, caprylic acid, capric acid, lauric acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof such as are obtained, for example, in the pressure hydrolysis of natural fats and oils. Technical soap mixtures based on C₁₂₋₁₈ or C₁₂₋₁₄ coconut oil fatty acid, or C₁₆₋₁₈ tallow fatty acid may also be used. In some embodiments, palm kemalate may also be used. It is envisioned that any combination of the soaps described herein may also be used to form the bar soap.

The amount of soap included in the composition will vary depending on the exact components chosen, the identity of other components employed, and the desired physical and performance characteristics to be achieved. Normally, however, the amount of soap in particularly useful embodiments will range from about 25 to about 85% by total weight of the bar soap. In some embodiments, the soap is present in an amount from about 40 to about 75% by total weight of the bar soap.

The fourth component used in forming the bar soap described herein includes water. In some embodiments the water may represent from about 1.0 to about 40% by total weight of the bar soap. Alternatively, the water may be present from about 5 to about 35% by total weight of the bar soap.

In some embodiments, emollients may be added to the four components described above. Emollients may include hydrophilic emollients, hydrophobic emollients, or a combination thereof. Useful hydrophilic emollients can include glycerin, propylene glycol or other water soluble or dispersible alcohols or polyols. Useful hydrophobic emollients can include fatty esters, triglycerides, silicones or other oil soluble or oil dispersible types of materials. In some embodiments, the emollient can be in the range of about 3 to about 50% by wt of the total composition. In other embodiments, the emollient can represent about 10 to about 40% by weight of the total composition.

In still other embodiments, the bar soap may include exfoliating agents. Suitable exfoliating agents include any agent capable of enhancing the soaps scrubbing ability to roughen the skin to allow the ingredients of the bar soap to experience greater skin surface interaction and deeper penetration into the affected skin layers. Some examples include, but are not limited to, hydrogenated fats, inorganic salts such as sodium citrate or relatively low-molecular weight organics, such as sugars; synthetic polymers such as polyethylene powders, polyethylene beads, polyethylene waxes and granulated particles or organo-polysiloxane; vegetable matter such as the endocarp of apricot, peach and walnut seeds, almond flesh, and wood flour. Additionally, birch powder or coconut pulp have been suggested as useful exfoliating agents. Other examples include the use of animal matter such as pearl dust or powdered crab shell and finely ground minerals such as, silica, pumice or limestone. Mineral exfoliants can also be used such as aluminium oxide, synthetic alumina, corundum, volcanic ash, diatomaceous earth, bentonite, and feldspar.

The exfoliating agents, i.e., polyethylene wax or beads, may be incorporated into the bar soap to assist in scrubbing the skin. The enhanced scrubbing will roughen the skin and increase the surface area with which the bar soap ingredients may interact. In addition, the exfoliating agents will create access to deeper layers of the skin, i.e., sub dermal layers thereby allowing the ingredients in the bar soap described herein to penetrate deeper into the skin. By accessing the deeper layers of the skin, the bar soap can be useful in treating dermatitis caused by uroshiol induced toxins even shortly after the uroshiol may have been absorbed by the body into the sub dermal layers of the skin. This may be particularly important when considering the average person experiencing dermatitis caused by uroshiol induced toxins does not normally seek treatment until experiencing the symptoms, i.e., rash, and itchy skin, which is the human body's reaction to the toxin being absorbed into the sub dermal layers of the skin.

Advantageously, active agents other than the emollients and exfoliating agents defined above may be added to the bar soap in a safe and effective amount. Illustrative examples of active ingredients include but are not limited to antimicrobial and antifungal actives, vitamins, anti-acne actives; anti-wrinkle, anti-skin atrophy and skin repair actives; skin barrier repair actives; non-steroidal cosmetic soothing actives; artificial tanning agents and accelerators; skin lightening actives; sunscreen actives; sebum stimulators; sebum inhibitors; anti-oxidants; protease inhibitors; skin tightening agents; anti-itch ingredients; hair growth inhibitors; 5-alpha reductase inhibitors; desquamating enzyme enhancers; anti-glycation agents; topical anesthetics, or mixtures thereof.

Active agents may be selected from water soluble active agents, oil soluble active agents, pharmaceutically-acceptable salts and mixtures thereof. The term “active agent” as used herein, means personal care actives which can be used to deliver a benefit to the skin and/or hair and which generally are not used to confer a conditioning benefit, as is conferred by emollients previously described herein. The term “safe and effective amount” as used herein, means an amount of active agent high enough to modify the condition to be treated or to deliver the desired skin care benefit, but low enough to avoid serious side effects. The term “benefit,” as used herein, means the therapeutic, prophylactic, and/or chronic benefits associated with treating a particular condition with one or more of the active agents described herein. What is a safe and effective amount of the active agent ingredient will vary with the specific active agent, the ability of the active to penetrate through the skin, the age, health condition, and skin condition of the user, and other like factors.

In some embodiments, the bars of the present disclosure may comprise from about 0.01% to about 10% by total weight of the bar soap of the active agent. In some embodiments, the bars of the present disclosure may comprise from about 1.0% to about 5% by total weight of the bar soap of the active agent.

Anti-acne actives can be effective in treating acne vulgaris, a chronic disorder of the pilosebaceous follicles. Nonlimiting examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, mixtures thereof and the like.

Antimicrobial and antifungal actives can be effective to prevent the proliferation and growth of bacteria and fungi. Nonlimiting examples of antimicrobial and antifungal actives include b-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, triclosan; triclocarban; and mixtures thereof and the like.

Anti-wrinkle, anti-skin atrophy and skin repair actives can be effective in replenishing or rejuvenating the epidermal layer. These actives generally provide these desirable skin care benefits by promoting or maintaining the natural process of desquamation. Nonlimiting examples of antiwrinkle and anti-skin atrophy actives include vitamins, minerals, and skin nutrients such as milk, vitamins A, E, and K; vitamin alkyl esters, including vitamin C alkyl esters; magnesium, calcium, copper, zinc and other metallic components; retinoic acid and its derivatives (e.g., cis and trans); retinal; retinol; retinyl esters such as retinyl acetate, retinyl palmitate, and retinyl propionate; vitamin B 3 compounds (such as niacinamide and nicotinic acid), alpha hydroxy acids, beta hydroxy acids, e.g. salicylic acid and derivatives thereof (such as 5-octanoyl salicylic acid, heptyloxy 4 salicylic acid, and 4-methoxy salicylic acid); mixtures thereof and the like.

Skin barrier repair actives are those skin care actives which can help repair and replenish the natural moisture barrier function of the epidermis. Nonlimiting examples of skin barrier repair actives include lipids such as cholesterol, ceramides, sucrose esters and pseudo-ceramides; ascorbic acid; biotin; biotin esters; phospholipids, mixtures thereof, and the like.

Non-steroidal cosmetic soothing actives can be effective in preventing or treating inflammation of the skin. The soothing active enhances the skin appearance benefits of the present invention, e.g., such agents contribute to a more uniform and acceptable skin tone or color. Nonlimiting examples of cosmetic soothing agents include the following categories: propionic acid derivatives; acetic acid derivatives; fenamic acid derivatives; mixtures thereof and the like.

Artificial tanning actives can help in simulating a natural suntan by increasing melanin in the skin or by producing the appearance of increased melanin in the skin. Nonlimiting examples of artificial tanning agents and accelerators include dihydroxyacetaone; tyrosine; tyrosine esters such as ethyl tyrosinate and glucose tyrosinate; mixtures thereof, and the like.

Skin lightening actives can actually decrease the amount of melanin in the skin or provide such an effect by other mechanisms. Nonlimiting examples of skin lightening actives useful herein include aloe extract, alpha-glyceryl-L-ascorbic acid, aminotyroxine, ammonium lactate, glycolic acid, hydroquinone, 4 hydroxyanisole, mixtures thereof, and the like.

Also useful herein are sunscreen actives. Nonlimiting examples of sunscreens which are useful in the compositions of the present invention are those selected from the group consisting of octyl methoxyl cinnamate (Parsol MCX) and butyl methoxy benzoylmethane (Parsol 1789), 2-ethylhexyl p-methoxycinnamate, 2-ethylhexyl N,N-dimethyl-p-aminobenzoate, p-aminobenzoic acid, 2-phenylbenzimidazole-5-sulfonic acid, oxybenzone, mixtures thereof, and the like.

Sebum stimulators can increase the production of sebum by the sebaceous glands. Nonlimiting examples of sebum stimulating actives include bryonolic acid, dehydroetiandrosterone (DHEA), orizanol, mixtures thereof, and the like.

Sebum inhibitors can decrease the production of sebum by the sebaceous glands. Nonlimiting examples of useful sebum inhibiting actives include aluminum hydroxy chloride, corticosteroids, dehydroacetic acid and its salts, dichlorophenyl imidazoldioxolan (available from Elubiol), mixtures thereof, and the like.

Also useful as actives in the present invention are protease inhibitors. Protease inhibitors can be divided into two general classes: the proteinases and the peptidases. Proteinases act on specific interior peptide bonds of proteins and peptidases act on peptide bonds adjacent to a free amino or carboxyl group on the end of a protein and thus cleave the protein from the outside. The protease inhibitors suitable for use in the present invention include, but are not limited to, proteinases such as serine proteases, metalloproteases, cysteine proteases, and aspartyl protease, and peptidases, such as carboxypepidases, dipeptidases and aminopepidases, mixtures thereof and the like.

Other useful as active ingredients in the present invention are skin tightening agents. Nonlimiting examples of skin tightening agents which are useful in the compositions of the present invention include monomers which can bind a polymer to the skin such as terpolymers of vinylpyrrolidone, (meth)acrylic acid and a hydrophobic monomer comprised of long chain alkyl (meth)acrylates, mixtures thereof, and the like.

Active ingredients in the present invention may also include anti-itch ingredients. Suitable examples of anti-itch ingredients which are useful in the compositions of the present invention include hydrocortisone, methdilizine and trimeprazineare, mixtures thereof, and the like.

Nonlimiting examples of hair growth inhibitors which are useful in the compositions of the present invention include 17 beta estradiol, anti angiogenic steroids, curcuma extract, cycloxygenase inhibitors, evening primrose oil, linoleic acid and the like. Suitable 5-alpha reductase inhibitors such as ethynylestradiol and, genistine mixtures thereof, and the like.

Nonlimiting examples of desquamating enzyme enhancers which are useful in the compositions of the present invention include alanine, aspartic acid, N methyl serine, serine, trimethyl glycine, mixtures thereof, and the like.

A nonlimiting example of an anti-glycation agent which is useful in the compositions of the present invention would be Amadorine (available from Bamet Products Distributor), and the like.

In addition, the bar soap may include 0 to 15% by total weight of the bar soap of the following optional ingredients: perfumes; sequestering agents, such as tetrasodium ethylenediaminetetraacetate (EDTA); chelating agents, such as disodium EDTA; coloring agents, opacifiers and pearlizers, such as zinc stearate, magnesium stearate, TiO₂, and the like; preservatives, such as quaternium-15, pentasodium pentate, and tetrasodium etidronate; viscofying agents, such as carbomer and C12-15 Pareth 9; skin-soothing agents, such as aloe vera, aloe barbadensis leaf juice, avena sative (oat) flour; and skin-healing agents, such as vitamin E oil, vitamin E acetate, and antioxidants; strong ionizing salts, such as sodium chloride; sud boosters, such as diethanol amides; all of which are useful in enhancing the appearance or cosmetic properties of the product.

The order of addition of the ingredients of the bar soap is not critical. The bar soaps according to the present disclosure may be produced by any methods known to those skilled in the art. Conventional processes for mixing or homogenizing, kneading, pilling, extruding, pelleting, milling, plodding, cutting and bar pressing are known to one skilled in the art and may be used for the production of the bar soaps according to the invention.

Once formed, the bar soap should be applied to the affected area as soon as possible after poison ivy or similar exposure is suspected. In applying the bar soap to the affected area, the bar soap should be wetted prior to application. The suspected areas of exposure should be washed with the wetted bar soap and rinsed following the application. These steps may be repeated as required to ensure removal of the uroshiol from the patients skin.

The bar soaps according to the present disclosure are distinguished by good foam stability, foaming power, excellent skin compatibility, and excellent ability to wash away uroshiol from a patients skin.

It is well understood that various modifications may be made to the embodiments disclosed herein. Therefore, the above description should not be construed as limiting, but merely as exemplifications of particularly useful embodiments. Those skilled in the art will envision other modifications within the scope and spirit of the claims appended hereto.

What is claimed is:

1. A bar soap comprising:
an alkyl phenol ethoxylate;
a sulfur-containing surfactant;
a soap; and
water.

2. The bar soap of claim 1 wherein the alkyl phenol ethoxylate is nonoxynol.

3. The bar soap of claim 1 wherein the sulfur-containing surfactant is selected from the group consisting of alkyl sulfates, sodium dodecyl sulfates, sodium lauryl sulfates, sulfoacetates, alkyl ether sulfates, oxyalkyl ether sulfates, linear alkylbenzene sulfonates, secondary alkane sulfonates, sulfosuccinates, sodium sulfosuccinates, disodium lauryl sulfosuccinates, disodium laureth sulfosuccinates, sodium coco sulfates, a-olefine sulfonates, sulfonated fatty-acids and combinations thereof.

4. The bar soap of claim 1 wherein the sulfur-containing surfactant is sodium lauryl sulfate.

5. The bar soap of claim 1 wherein the soap is selected from the group consisting of sodium tallowate, sodium cocoate, palm kemalate, sodium palmitate and combinations thereof.

6. The bar soap of claim 1 wherein the soap is a compound of the formula:
R¹CO—ONa
in which R¹CO is an aliphatic acyl radical containing 4 to 22 carbon atoms are suitable for use in accordance with the invention.

7. The soap bar of claim 1 wherein the water is present from about 5 to about 35% by weight of the soap bar.

8. The soap bar of claim 1 further comprising an emollient selected from the group consisting of hydrophilic emollients, hydrophobic emollients, and combinations thereof.

9. The soap bar of claim 8 wherein the emollient is glycerin.

10. The soap bar of claim 1 further comprising an active agent.

11. The soap bar of claim 10 wherein the active agent is selected from the group consisting of aloe vera, avena satis flour, aloe barbadensis leaf juice, vitamin E and combinations thereof.

12. The soap bar of claim 1 further comprising at least one optional ingredient.

13. The soap bar of claim 1 further comprising at least one exfoliating agent.

14. The soap bar of claim 13 wherein the exfoliating agent is polyethylene wax, polyethylene beads, polyethylene granules and combinations thereof.

15. A method of washing away at least one of poison ivy, oak and sumac comprising:
preparing a bar soap comprising an alkylphenol ethoxylate, a sulfur-containing surfactant, a soap and water,
washing the affected area using the bar soap; and
rinsing the affected area.

16. The method of claim 15 wherein preparing the bar soap further includes adding glycerin.

17. The method of claim 15 wherein preparing the bar soap further includes adding an ingredient selected from the group consisting of exfoliating agents, fillers, colors, preservatives, active agents, ionizing salts and combinations thereof.
